(19) United States
(12) Patent Application Publication
(10) Pub. No.: US 2005/0255175 A1
(43) Pub. Date: Nov. 17, 2005

(54) Method for protecting nephron against injury caused by disruption of a chemical environment in the kidneys

(76) Inventor: W. Patrick Burgess, Charlotte, NC (US)

Correspondence Address: SCHWARTZ LAW FIRM, P.C., 6100 FAIRVIEW ROAD, SUITE 530, CHARLOTTE, NC, 28210, US

(21) Appl. No.: 10/847,482

(22) Filed: May 17, 2004

(51) Int. Cl. A61K 33/42

(52) U.S. Cl. 424/717

(57) ABSTRACT

A method is provided for protecting nephrons against injury caused by disruption of a chemical environment in the kidneys. The method includes the step of administering a prophylactic alkaline solution into the body of a subject. The solution has a concentration of bicarbonate greater than 70 mEq/L. The method protects the nephrons by administering a prophylactic solution into the body to counter an effect of the kidneys' acid-base balance on an anticipated disruption of the kidney function.

### TECHNICAL FIELD AND BACKGROUND OF THE INVENTION

This invention relates generally to a method for protecting nephrons against injury caused by disruption of a chemical environment in the kidneys. The method protects the nephrons by administering a prophylactic solution into the body to counter an effect of the kidneys' acid-base balance on an anticipated disruption of the kidney function. In one particular application, the invention is a method of administering a contrast medium in a manner which reduces the incidence of contrast-induced nephropathy (CIN).

The kidneys' main function is to eliminate excess fluid and waste material from the blood. When the kidneys lose this filtering ability, dangerous levels of fluid and waste accumulate in the body causing kidney (renal) failure. Acute kidney failure is most likely to happen after complicated surgery or severe injuries, or when blood vessels leading to the kidneys become blocked or experience low blood pressure, or when the kidneys are exposed to chemical compounds that are potentially toxic. Conversely, chronic kidney failure usually develops slowly with few symptoms in the early stages. Many people with chronic kidney failure have no symptoms until their kidney function has decreased to less than 25 percent of normal. High blood pressure and diabetes are the most common causes of chronic kidney failure.

A single adult kidney contains roughly a million nephrons, each consisting of a tuft of capillary blood vessels (glomerulus) and tubules that lead to the collecting system. Each tuft of capillaries filters fluid from the bloodstream, and passes it to a tubule. The filtered fluid contains both waste products and substances vital for health. From the tubules, waste byproducts such as urea, uric acid and creatinine are excreted in urine while substances the body needs such as sugar, protein, calcium and salts are absorbed back into the bloodstream. While this filtration system is generally able to clear all the waste products produced by the body, problems can occur if the tubules or glomeruli are damaged or diseased.

Many conditions and circumstances can damage kidneys, including intrinsic kidney disease or injury, high blood pressure, diabetes mellitus, exposure to toxins and certain medications, kidney stones, tumors and even infections in other parts of your body. Many of these may show no signs or symptoms until irreparable damage has occurred.

The mechanism of the vast majority of acute kidney damage and injury is modulated by the intra-kidney formation of free radicals known to be increased in an acid environmental (pH that is low), as compared to normal body pH. One of the functions of the kidney is to regulate acid-base metabolism by actively absorbing the filtered bicarbonate and generating bicarbonate, while excreting the typical acid load of subjects. This process of eliminating the “acid load” causes the generation of renal tubular fluid which is relatively acidic compared to normal tissue. This acid environment accelerates the formation of free radicals under certain conditions. Existing medical references support the attenuation of free radical formation by inducing a more normal pH environment in the kidney.

The use of iodinated, radiographic contrast (RC) media has long been recognized as a contributing factor in acute kidney dysfunction. Examples of imaging medical procedures requiring the use of RC include CT scan enhancement, arteriograms, cardiac catherization, vascular studies, stents, lumbar myelography, thoraco-cervical myelography, cerebral angiography, peripheral arteriography, venography, angiocardiography, left ventriculography, selective visceral arteriography, digital subtraction abgiography, urography, arthrography, and computer tomography enhancement. The degree of acute kidney dysfunction—labeled “contrast induced nephropathy” (CIN)—ranges from a short-term slight increase in serum creatinine levels to overt kidney failure requiring temporary or permanent dialysis, and in some cases resulting in death. CIN is broadly defined as a rise in serum creatinine levels in relation to the administration of contrast media. CIN has been reported to be the third most common cause of kidney insufficiency occurring in hospitalized patients, and it might be a factor in up to 10 percent of all cases of acute kidney failure.

Prior art efforts to treat kidney dysfunction recognize the use of a sodium bicarbonate infusion. These existing methods, however, are fraught with complications, drawbacks, and inconclusive test results.

Complications of Bicarbonate Infusions

Hypertonic solutions of sodium bicarbonate (7.5% or 1 M) are supplied in most hospitals in 50 ml ampules to be administered slowly, or added to other intravenous solutions. Rapid infusions or excessive volumes of this hypertonic solution are known to cause serious injury. This injury can occur in the form of a rapid depression of serum potassium and subsequent cardiac rhythm disturbances (even fatal disturbances), depression of serum ionized calcium with an associated drop in blood pressure, and severe pain and tissue necrosis at the site of an intravenous extravagation.

If sodium bicarbonate is prepared in a solution of 25 to 50 mEq/L, as directed in prior U.S. Pat. No. 5,112,622, then the volume required to reach a target level of 2.0 mEq/kg (of subject weight) in a 70 kg subject would be 5.6 to 2.8 L. This volume is large and difficult for normal subjects to handle in an eight hour period, but entirely unacceptable as an administered dose of fluids to “sick” or elderly subjects undergoing a substantial medical procedure, such as cardiac catheterization or cardiac surgery. The critical care literature is full of references to the danger of sodium bicarbonate infusions in critically ill subjects.

The bicarbonate pretreatment of the present invention has been shown to nearly eliminate acute kidney failure associated with contrast exposure. Similar evidence shows that similar doses can reduce the acute kidney failure associated with cardiovascular surgery. The choice of bicarbonate concentration in the 150 to 300 mEq/L range allows an effective dose of solution to be administered in a volume of fluid that is well tolerated by “sick” subjects undergoing the diagnostic procedure.

Since many other causes of kidney dysfunction (other than CIN) are induced by the free radical formation process, the present method can also be expected to respond favorably against these disruptions by similar administration of bicarbonate, provided excessive volume and excessive concentration can be avoided. Suggested prior art treatments using sodium bicarbonate, such as described in the '622 patent, would require a volume of fluid in excess of what even a normal subject could be expected to tolerate without death or serious complications. The present invention has identified an effective and tolerable dose of the bicarbonate anion, concentration of the anion, and timely administration necessary for a successful prophylaxis.

### SUMMARY OF INVENTION

Therefore, it is an object of the invention to provide a method for protecting nephrons against injury caused by disruption of the chemical environment in the kidneys.

It is another object of the invention to provide a method for protecting nephrons against injury caused by administration of iodinated, radiographic contrast (RC) media.

It is another object of the invention to provide a method for administering a contrast medium which substantially eliminates the incidence of contrast-induced nephropathy (CIN).

It is another object of the invention to provide a method for administering a contrast medium which reduces the subject's time in the hospital by 6 hours or more, as compared to other treatments which infuse saline solution for 12 hours before and 12 hours after the contrast exposure.

It is another object of the invention to provide a method for administering a contrast medium which utilizes a renoprotective bicarbonate solution received intravenously or orally in pill form.

It is another object of the invention to provide a method for administering a contrast medium which is especially applicable to chronically ill subjects who currently may not receive a contrast medium for fear of inducing CIN.

It is another object of the invention to provide a method for administering a contrast medium in a manner which will improve a healthcare institution's rating for insurance and referral purposes.

It is another object of the invention to provide a method for administering a contrast medium in a manner which reduces the physician's exposure to legal liability.

It is another object of the invention to provide a method for administering a contrast medium in a manner which will reduce overall healthcare costs.

It is another object of the invention to provide a method for administering a contrast medium in a manner which will not damage or impair healthy kidneys.

It is another object of the invention to provide a method for administering a contrast medium in a manner which is easily administered by the hospital staff.

It is another object of the invention to provide a method for administering a contrast medium which is applicable to both ionic and non-ionic contrast media.

These and other objects of the present invention are achieved in the preferred embodiments disclosed below by providing a method for protecting nephrons against injury caused by disruption of a chemical environment in the kidney. The method includes the step of administering a prophylactic alkaline solution into the body of a subject. The solution has a concentration of bicarbonate greater than 70 mEq/L. When administered orally in pill form, the concentration of bicarbonate is greater than 11.9 mEq/gram.

Preferably, the prophylactic solution has a bicarbonate concentration within a range of 100 mEq/L and 300 mEq/L. The bicarbonate is administered into the body at a dose of between 0.8 and 5.0 mEq of bicarbonate per kg of subject weight, and more preferably, between 1.2 and 3.0 mEq of bicarbonate per kg of subject weight.

According to another preferred embodiment of the invention, the prophylactic solution contains sodium.

According to another preferred embodiment of the invention, the prophylactic solution contains potassium.

Preferably, the prophylactic solution contains between 75%-100% sodium and between 25%-0% potassium.

According to another preferred embodiment of the invention, the total volume of prophylactic solution administered into the body ranges from 2.0 to 15.0 ml/kg (of subject weight).

According to another preferred embodiment of the invention, the prophylactic solution is administered into the body at a rate of between 0.5 and 2.5 ml/kg (of subject weight)/hr.

In another embodiment, the invention is a method for reducing contrast-induced nephropathy resulting from administration of a contrast medium into the body of a subject. The method includes the steps of, prior to receiving the contrast medium, administering an initial dose of prophylactic alkaline solution into the body. The solution has a concentration of bicarbonate greater than 70 mEq/L. After receiving the contrast medium, a maintenance dose of the prophylactic solution is administered into the body.

According to another preferred embodiment of the invention, the maintenance dose is administered into the body at a rate lower than that of the initial dose.

According to another preferred embodiment of the invention, the initial dose of prophylactic solution is administered into the body at a time beginning no later than 3 hours and no sooner than 15 minutes prior to receiving the contrast medium.

According to another preferred embodiment of the invention, the maintenance dose is administered into the body for a period of between 5 and 12 hours after receiving the contrast solution.

In yet another embodiment, the invention is a method for administering a contrast medium into the body of a subject. The method includes the steps of, prior to receiving the contrast medium, administering an initial dose of prophylactic alkaline solution into the body. The solution has a concentration of bicarbonate greater than 70 mEq/L. The contrast medium is then administered into the body. After receiving the contrast medium, a maintenance dose of the prophylactic solution is administered into the body.

### DESCRIPTION OF THE PREFERRED EMBODIMENT AND BEST MODE

The invention is a method for protecting nephrons against injury caused by a disruption of a chemical environment in the kidney. Such disruptions commonly result from exposure to iodinated radiographic contrast media (administered prior to CT scans); emergency exploratory surgeries; shock; trauma; certain transplant immunosuppression regimens (e.g., cyclosporine and Prograf®); certain antibiotic therapy (gentamicin, tobramicin, amikacin, vancomycin); and NSAID's (Motrin®, Indocin®, tordol, Advil®, naprasyn). In its broadest application, the concept of the present method is applicable for protecting any organ by manipulating the body's acid-base balance through infusion of a prophylactic alkaline solution in a prescribed concentration and dosage.

The alkaline solution is contained inside a transparent sterile bag or other sterile container, and is administered into the body either intravenously or orally. In the example described below, the method is a prophylactic treatment for preventing kidney damage resulting from exposure to contrast media. Examples of such iodinated radiographic contrast media include iopamidol (a tri-iodinated, non-ionic, water-soluble, contrast medium), iomeprol, iohexol, iobitridol, iodixanol, ioversol, ioxaglate, iotrolan, iopromide, iobitridol, and diatrizoate. The treatment conditions the nephrons in the kidney to become more alkaline in order to better tolerate stress caused by the iodinated contrast media. By this method, the goal is to provide between 25% and 40% of the total dose of the prophylactic solution over a time period beginning at least 15 minutes prior to receiving the contrast medium. The remainder of the total dose of the solution is administered during IV infusion of the contrast medium and over a subsequent 5 to 12 hour period. The total dose is preferably between 1.2 and 3.0 mEq of bicarbonate per kilogram of subject weight.

According to one embodiment, the solution comprises a bicarbonate anion combined with a sodium and/or potassium cation. The cation is preferably a mixture of sodium (75% to 100%, typically 240 mEq/L) and potassium (25% to 0%, typically 10 mEq/L). The preferred concentration of the bicarbonate anion is greater than 70 mEq/L, and more preferably, between 100 mEq/L and 300 mEq/L. Contrary to the teachings of the prior art, a higher bicarbonate concentration is desirable in order to infuse the necessary chemicals without flooding the body with an excessive volume of fluid. Ideally, the concentration should be around 200 to 300 mEq/L, so that the total volume infused is from 2.0 to 15.0 ml/kg (of subject weight). For a goal infusion of 1.8 mEq/kg (of subject weight) using 250 mEq/L solution, the volume infused over a 7 to 8 hour period is 7.3 ml/kg (of subject weight)—or an average of 1 ml/kg (of subject weight)/hour.

### EXAMPLE

A prophylactic solution consisting of 5.5 grams of sodium, 0.39 grams of potassium, 15.3 grams of bicarbonate, and 978.8 grams of water per liter is prepared and administered into the body via an IV infusion. The treatment is provided as follows:
(a) at least one hour prior to (but not more than three hours before) intravenous infusion of the iodinated contrast medium, infuse an initial bolus dose of prophylactic solution at a rate of 2.0 mL per kilogram of subject weight over one hour;
(b) after the initial bolus infusion, reduce the infusion rate to 0.62 mL per kilogram of subject weight per hour, and continue infusion throughout administration of the contrast medium and for six (6) hours after the contrast administration is completed;
(c) monitor subject during the infusion period for occurrence of adverse symptoms or signs including severe hypertension, pulmonary edema, decompensated heart failure, shortness of breath and wheezing; and
(d) upon occurrence of adverse symptoms or signs, reduce the infusion rate to 10 mL per hour and seek medical evaluation for the subject.

The following Table indicates the prescribed subject dosage rate for the initial bolus and maintenance infusion:

| | | Initial Bolus | Maintenance |
|---|---|---|---|
| Subject Weight | | mL | Infusion Rate, |
| kilograms, kg | pounds, lbs | over 1 hour | mL per hour |
| <32.8 kg | <72.2 lbs | 2 X wt(kg) | 0.62 X wt(kg) |
| 32.8 to 40.8 kg | 72.2 to 89.8 lbs | 81 mL | 25 mL/hr |
| 40.8 to 48.8 kg | 89.8 to 107.5 lbs | 97 mL | 30 mL/hr |
| 48.9 to 56.9 kg | 107.6 to 125.2 lbs | 113 mL | 35 mL/hr |
| 57.0 to 64.9 kg | 125.3 to 142.9 lbs | 129 mL | 40 mL/hr |
| 65.0 to 73.0 kg | 143.0 to 160.7 lbs | 145 mL | 45 mL/hr |
| 73.1 to 81.0 kg | 160.8 to 178.4 lbs | 161 mL | 50 mL/hr |
| 81.1 to 89.1 kg | 178.5 to 196.2 lbs | 177 mL | 55 mL/hr |
| 89.2 to 97.2 kg | 196.3 to 213.9 lbs | 194 mL | 60 mL/hr |
| 97.3 to 105.2 kg | 214.0 to 231.6 lbs | 210 mL | 65 mL/hr |
| 105.3 to 113.3 kg | 231.7 to 249.4 lbs | 226 mL | 70 mL/hr |
| 113.4 to 121.4 kg | 249.5 to 267.1 lbs | 242 mL | 75 mL/hr |
| 121.5 to 129.4 kg | 267.2 to 284.9 lbs | 258 mL | 80 mL/hr |
| 129.5 to 137.5 kg | 285.0 to 302.6 lbs | 274 mL | 85 mL/hr |
| 137.6 to 145.6 kg | 302.7 to 320.4 lbs | 290 mL | 90 mL/hr |
| 145.7 to 153.6 kg | 320.5 to 338.1 lbs | 306 mL | 95 mL/hr |
| 153.7 to 161.7 kg | 338.2 to 355.8 lbs | 323 mL | 100 mL/hr |
| >161.7 kg | >355.8 lbs | 2 Xwt(kg) | 0.62 Xwt(kg) |

A method for protecting nephrons against injury caused by a disruption of the chemical environment in the kidney, a method for reducing contrast-induced nephropathy, and a safe method for administering a contrast medium into the body of a subject are described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

1. A method for protecting nephrons against injury caused by disruption of a chemical environment in the kidney, said method comprising the step of administering a prophylactic alkaline solution into the body of a subject, the solution comprising a concentration of bicarbonate greater than 70 mEq/L.

2. A method according to claim 1, wherein the prophylactic solution comprises a bicarbonate concentration within a range of 100 mEq/L and 300 mEq/L.

3. A method according to claim 1, wherein the prophylactic solution is administered into the body at a dose of between 0.8 and 5.0 mEq of bicarbonate/kg (of subject weight).

4. A method according to claim 1, wherein the prophylactic solution comprises sodium.

5. A method according to claim 1, wherein the prophylactic solution comprises potassium.

6. A method according to claim 1, wherein the prophylactic solution comprises between 75%-100% sodium and between 25%-0% potassium.

7. A method according to claim 1, wherein the total volume of prophylactic solution administered into the body ranges from 2.0 to 15.0 ml/kg (of subject weight).

8. A method according to claim 1, wherein the prophylactic solution is administered into the body at a rate of between 0.5 and 2.5 ml/kg (of subject weight)/hr.

9. A method for reducing contrast-induced nephropathy resulting from administration of a contrast medium into the body of a subject, said method comprising the steps of:
(a) prior to receiving the contrast medium, administering an initial dose of prophylactic alkaline solution into the body, the solution comprising a concentration of bicarbonate greater than 70 mEq/L; and
(b) after receiving the contrast medium, administering a maintenance dose of the prophylactic solution into the body.

10. A method according to claim 9, wherein the maintenance dose is administered into the body at a rate lower than that of the initial dose.

11. A method according to claim 9, wherein the initial dose of prophylactic solution is administered into the body at a time beginning no later than 3 hours and no sooner than 15 minutes prior to receiving the contrast medium.

12. A method according to claim 9, wherein the maintenance dose is administered into the body for a period of between 5 and 12 hours after receiving the contrast solution.

13. A method according to claim 9, wherein the prophylactic solution comprises a bicarbonate concentration within a range of 100 mEq/L and 300 mEq/L.

14. A method according to claim 9, wherein the total volume of prophylactic solution administered into the body ranges from 2.0 to 15.0 ml/kg (of subject weight).

15. A method according to claim 9, wherein the prophylactic solution is administered into the body at a rate of between 0.5 and 2.5 ml/kg (of subject weight)/hr.

16. A method according to claim 9, wherein the prophylactic solution is administered into the body at a dose of between 0.8 and 5.0 mEq of bicarbonate/kg (of subject weight).

17. A method for administering a contrast medium into the body of a subject, comprising the steps of:
(a) prior to receiving the contrast medium, administering an initial dose of prophylactic alkaline solution into the body, the solution comprising a concentration of bicarbonate greater than 70 mEq/L;
(b) administering the contrast medium into the body; and
(c) after receiving the contrast medium, administering a maintenance dose of the prophylactic solution into the body.

18. A method according to claim 17, wherein the initial dose of prophylactic solution is administered into the body at a time beginning no later than 3 hours and no sooner than 15 minutes prior to receiving the contrast medium.

19. A method according to claim 17, wherein the maintenance dose is administered into the body for a period of between 5 and 12 hours after receiving the contrast solution.

20. A method according to claim 17, wherein the prophylactic solution comprises a bicarbonate concentration within a range of 100 mEq/L and 300 mEq/L.

21. A method according to claim 17, wherein the total volume of prophylactic solution administered into the body ranges from 2.0 to 15.0 ml/kg (of subject weight).

22. A method according to claim 17, wherein the prophylactic solution is administered into the body at a rate of between 0.5 and 2.5 ml/kg (of subject weight)/hr.

23. A method according to claim 17, wherein the prophylactic solution is administered into the body at a dose of between 0.8 and 5.0 mEq of bicarbonate/kg (of subject weight).
